(19) United States
(12) Patent Application Publication
(10) Pub. No.: US 2015/0202149 A1
(43) Pub. Date: Jul. 23, 2015

(54) PHARMACEUTICAL COMPOSITIONS OF MESALAMINE SUPPOSITORIES

(71) Applicant: CADILA HEALTHCARE LIMITED, AHMEDABAD (IN)

(72) Inventors: Nilendu SEN, Thane (IN); Sandhya Rajendra SHENOY, Thane (IN); Imran Shakoor GHOGARI, Thane (IN)

(73) Assignee: CADILA HEALTHCARE LIMITED

(21) Appl. No.: 14/600,373

(22) Filed: Jan. 20, 2015

(30) Foreign Application Priority Data
Jan. 21, 2014 (IN) 205/MUM/2014

(51) Int. Cl. A61K 9/02; A61K 31/196

(52) U.S. Cl. CPC A61K 9/02; A61K 31/196

(57) ABSTRACT

The present invention relates to pharmaceutical compositions of mesalamine suppositories. In particular, the invention relates to pharmaceutical suppositories comprising mesalamine or salts thereof and at least two oily or fatty bases. The invention also relates to processes for the preparation of such compositions and use thereof for treatment of ulcerative colitis or ulcerative proctitis.

### FIELD OF THE INVENTION

The present invention relates to mesalamine suppositories. In particular, the invention relates to pharmaceutical compositions of suppositories comprising mesalamine or pharmaceutically acceptable salts. The invention also relates to processes for the preparation of such compositions and use thereof for treating ulcerative colitis and ulcerative proctitis.

### BACKGROUND OF THE INVENTION

Inflammatory bowel disease (IBD) is the general name for diseases that cause inflammation in the small intestine and colon. Ulcerative colitis (UC) is the most common inflammatory bowel disease and it affects various portions of the gastrointestinal (GI) tract, particularly the lower GI tract, and more particularly the colon and/or rectum. A second IBD is Crohn's disease, which predominates in the small intestine (ileum) and the large intestine (colon).

Ulcerative colitis can be difficult to diagnose in that its symptoms are similar to other intestinal disorders and to Crohn's disease. Crohn's disease differs from ulcerative colitis because it causes deeper inflammation into the intestinal wall. Also, Crohn's disease usually occurs in the small intestine, although it can also occur in the mouth, esophagus, stomach, duodenum, large intestine, appendix, and anus.

Ulcerative colitis may occur in people of any age, but most often it starts between ages 15 and 30, or less frequently between ages 50 and 70. Children and adolescents sometimes develop this disease. Ulcerative colitis affects men and women equally and appears to run in some families.

Mesalamine, 5-aminosalicylic acid (5-ASA), is often used to treat UC and is effective in reducing disease symptoms and the incidence of relapse in UC. While mesalamine is available in oral form, intrarectal administration of it has several advantages. For example, rectal administration of a drug avoids some side-effects, such as gastrointestinal disorders, due to oral administration. As mesalamine is a locally GI active drug, lower doses of the drug can be administered rectally to obtain a better or equivalent therapeutic effect as that attained with a higher dose oral formulation. The absorption of a drug orally administered may also be affected by whether it is administered before or after each meal or between meals.

There is no such food effect when drugs are administered intrarectally. Intrarectal administration can be performed even during nausea, vomiting or unconsciousness, or after surgical operation.

1 g mesalamine suppository is currently marketed in the U.S. by Forest Labs Inc. as CANASA® for the treatment of active ulcerative proctitis.

U.S. Pat. Nos. 8,217,083; 7,541,384; 8,436,051 disclose mesalamine rectal suppositories comprising mesalamine and an oily or a fatty base, wherein the mesalamine has a tap density ranging from about 600 to about 800 g/L and the suppository has a drug load ranging from 35% to 50%.

Hence, there still remains a need for alternative pharmaceutical suppositories comprising mesalamine in order to achieve desired dissolution profile of the compositions with comfort in application.

### SUMMARY OF THE INVENTION

In one general aspect there is provided a pharmaceutical suppository comprising mesalamine or salts thereof and at least two oily or fatty bases.

In another general aspect of the present invention to provide pharmaceutical suppositories comprising mesalamine or salts thereof and at least two oily or fatty bases, wherein the mesalamine has a tap density ranging from about 250 g/L to about 580 g/L (as measured by USP <616>).

It is another aspect of the present invention to provide pharmaceutical suppositories comprising mesalamine or salts thereof and at least two oily or fatty bases, wherein the mesalamine has a tap density ranging from about 250 g/L to about 580 g/L (as measured by USP <616>), wherein the drug load of the suppository is not more than 35% w/w.

It is another aspect of the present invention to provide pharmaceutical suppositories comprising mesalamine or salts thereof and at least two oily or fatty bases, wherein the mesalamine has a tap density ranging from about 250 g/L to about 580 g/L (as measured by USP <616>), preferably the mesalamine has a tap density ranging from about 350 g/L to about 550 g/L (as measured by USP <616>), wherein the drug load of the suppository is not more than 35% w/w.

It is another aspect of the present invention to provide pharmaceutical suppositories comprising mesalamine or salts thereof and at least two oily or fatty bases, wherein the mesalamine has a tap density ranging from about 250 g/L to about 580 g/L (as measured by USP <616>), preferably about 350 g/L to about 550 g/L, more preferably about 400 g/L to about 500 g/L, wherein the suppository may include from about 400 to about 1600 mg mesalamine.

It is another aspect of the present invention to provide pharmaceutical suppositories comprising mesalamine or salts thereof and at least two oily or fatty bases, wherein each suppository base is having an ascending melting point of not more than 37° C.

It is further aspect of the present invention to provide pharmaceutical suppositories, releases at least about 75% by weight of the mesalamine contained in the suppository within 2 hours of dissolution as measured With USP Apparatus #2 at 40° C., a paddle rotation speed of 125 rpm, and 3 sinker turns in 0.2 M phosphate buffer at a pH of 7.5.

It is further aspect of the present invention to provide a method of preparing a mesalamine rectal suppository by preparing the suppository from mesalamine having a tap density ranging from about 250 g/L to about 580 g/L, more preferably about 350 g/L to about 500 g/L, with at least two oily or fatty bases, such as a hard fat, having an ascending melting point of not more than 37° C.

It is further aspect of the present invention to provide a method of treating ulcerative colitis or ulcerative proctitis, in a patient in need thereof by administering to the patient a mesalamine rectal suppository of the present invention. Preferably, the mesalamine suppository is administered once a day and more preferably once a day at bedtime.

The details of one or more embodiments of the present invention are set forth in the description below. Other features, objects and advantages of the invention will be apparent from the description.

### DETAILED DESCRIPTION OF THE INVENTION

It has been observed that generally when the drug load of a mesalamine suppository is increased, so too is the viscosity of the molten suspension which is cast to form the suppository. If the viscosity of the mesalamine suspension is too high, it cannot be cast into a suppository having acceptable content uniformity and good therapeutic properties. The inventors have surprisingly found that the viscosity of the measlamine suspension can be decreased by using combination of at least two oily or fatty bases having an ascending melting point of not more than 37° C. Further, the mesalamine has a tap density ranging from about 250 to about 580 g/L (as measured by USP <616>)

The inventors have further discovered that the present invention provides a better dissolution profile of mesalamine (a poorly soluble drug) from a suppository if mesalamine has a tap density ranging from about 250 to about 580 g/L (as measured by USP <616>) is used in with the combination of at least two oily or fatty bases having an ascending melting point of not more than 37° C.

According to one embodiment, the mesalamine has the following particle size distribution: ×10 is not more than 10 μm, ×50 is not more than 50 μm, and ×90 is not more than 100 μm.

The present invention provides provide pharmaceutical suppositories comprising mesalamine or salts thereof and at least two oily or fatty base, wherein the mesalamine has a tap density ranging from about 250 to about 580 g/L (as measured by USP <616>), preferably about 350 g/L to about 550 g/L, more preferably about 400 g/L to about 500 g/L, wherein the drug load of the suppository is not more than 35% w/w.

The present invention further provides pharmaceutical suppositories comprising mesalamine or salts thereof and at least two oily or fatty bases, wherein the mesalamine has a tap density ranging from about 250 to about 580 g/L (as measured by USP <616>), preferably about 350 g/L to about 550 g/L, more preferably about 400 g/L to about 500 g/L, wherein the suppository may include from about 850 to about 1150 mg mesalamine, and preferably includes about 950 mg to about 1050 mg mesalamine, and even more preferably about 1000 mg mesalamine.

It is an embodiment of the present invention to provide pharmaceutical suppositories comprising mesalamine or salts thereof and at least two oily or fatty bases, wherein the mesalamine has a tap density ranging from about 250 to about 580 g/L (as measured by USP <616>), preferably about 350 g/L to about 550 g/L, more preferably about 400 g/L to about 500 g/L, wherein the suppository includes from about 400 to about 600 mg mesalamine, and preferably includes about 450 to about 550 mg mesalamine, and even more preferably about 500 mg mesalamine.

One embodiment of the present invention is a mesalamine suppository comprising mesalamine and one or more pharmaceutically acceptable excipients, wherein the drug load of the suppository is not more than 35% w/w and preferably the drug load ranges from about 33% to about 35% w/w. The suppository may include from about 850 to about 1150 mg mesalamine, and preferably includes about 950 mg to about 1050 mg mesalamine (and even more preferably about 1000 mg mesalamine).

According to another embodiment, the suppository includes from about 400 to about 600 mg mesalamine, and preferably includes about 450 to about 550 mg mesalamine (and even more preferably about 500 mg mesalamine).

According to yet another embodiment, the suppository includes from about 1400 to about 1600 mg mesalamine, and preferably includes about 1450 to about 1550 mg mesalamine (and even more preferably about 1500 mg mesalamine). The mesalamine suppository may further include at least two oily or fatty bases, such as hard fat (e.g., hard fat NF).

In the framework of the present description “hard fat” as used herein means a mixture of monoglyceride, diglyceride and triglyceride of straight-chain saturated fatty acids containing 8 to 18 carbon atoms, and examples of such hard fat are mentioned in the literature, e.g. Martindale The Extra Pharmacopeia (28th edition, Page 1067, The Pharmaceutical Press, London, 1982) and Standards for Ingredients of Drugs not in the Japanese Pharmacopeia (Edited by Pharmaceutical Affairs Bureau., Ministry of Health and Welfare in Japan, Page 243, Jun. 28, 1993, Yakugyo Jiho Co., Ltd., Tokyo, Japan). Such hard fats are commercially available, for example under the trade names of Suppocire™A, Suppocire™AIML, Suppocire™AM, Suppocire™AML, Suppocire™AP, Suppocire™AS2, Suppocire™AS2X, Suppocire™ NA, Suppocire™NA0, Suppocire™ NA15, Suppocire™NAI 25 A, Suppocire™NAI 50, Suppocire™NAIS 90, Suppocire™NAS 50, and Suppocire™NAS 55 (manufactured by Gattefosse Inc.), “Isocacao” (manufactured by Kao Corp.), Witepsol™ H-5, Witepsol™ H-15, Witepsol™ H-35, Witepsol™ W-25, Witepsol™ W-35, Witepsol™ S-55 and Witepsol™ S-58 (all manufactured by Huls AG), Nissan Pharmasol™ B-115 and Nissan Pharmasol™ N-145 (all from Nippon Oil & Fats Co., Ltd.), etc.

According to a preferred embodiment, the mesalamine in the aforementioned suppositories is dispersed in a low melting suppository base (i.e., a suppository base having an ascending melting point of no more than 37° C.). A preferred low melting suppository base is hard fat having an ascending melting point of 32 to 35.5° C. More preferably, suitable low melting suppository base is hard fat having an ascending melting point of 33 .5 to 35.5° C. The dispersion is preferably substantially homogenous.

In another embodiment, the oily or fatty bases used in present invention are combination of at least two oily or fatty bases. Most preferable combination is Witepsol™ H-15 and Suppocire™NA15. The weight ratio of Witepsol™ H-15 to Suppocire™NA15 preferably in ratio from about 1:3 to about 3:1.

In an embodiment, the aforementioned suppositories each release at least about 75% by weight of the mesalamine contained in the suppository containing from about 850 to about 1150 mg mesalamine is within 2 hours of dissolution as measured with USP Apparatus #2 at 40° C., a paddle rotation speed of 125 rpm, and from 2 to 8 sinker turns in 0.2 M phosphate buffer at a pH of 7.5. The mesalamine in each of the aforementioned suppositories preferably has a tap density ranging from about 250 to about 580 g/L (as measured by USP <616>), preferably about 350 g/L to about 550 g/L, more preferably about 400 g/L to about 500 g/L and comprises combination of two hard fat bases.

In another embodiment, the aforementioned suppositories each release at least about 75% by weight of the mesalamine contained in the suppository containing from about 400 to about 600 mg mesalamine is within 2 hours of dissolution as measured with USP Apparatus #2 at 37° C., a paddle rotation speed of 75 rpm, and from 2 to 8 sinker turns in 0.2 M phosphate buffer at a pH of 7.5. The mesalamine in each of the aforementioned suppositories preferably has a tap density ranging from about 250 to about 580 g/L (as measured by USP <616>), preferably about 350 g/L to about 550 g/L, more preferably about 400 g/L to about 500 g/L and comprises combination of two hard fat bases.

In another embodiment, the aforementioned suppositories each release at least about 75% w/w by weight of the mesalamine contained in the suppository containing from about 1400 to about 1600 mg mesalamine is within 2 hours of dissolution as measured with USP Apparatus #2 at 40° C., a paddle rotation speed of 125 rpm, and from 2 to 8 sinker turns in 0.2 M phosphate buffer at a pH of 7.5. The mesalamine in each of the aforementioned suppositories preferably has a tap density ranging from about 250 to about 580 g/L (as measured by USP <616>), preferably about 350 g/L to about 550 g/L, more preferably about 400 g/L to about 500 g/L and comprises combination of two hard fat bases.

Yet another embodiment is a method of preparing a mesalamine rectal suppository by (A) providing a mesalamine rectal suppository and (B) measuring the dissolution rate of the suppository with USP Apparatus #2 at 40° C. and a paddle rotation speed of 125 rpm in 0.2 M phosphate buffer at a pH of 7.5. A sinker can be coiled around the suppository, for example, for 2 to 8 turns of wire (e.g., wire helix).

According to one embodiment, the mesalamine suppository is prepared by (A) melting the combination of suppository base, e.g., to form a molten solution, (B) adding mesalamine to the melted suppository base, and (C) molding the mixture.

Yet another embodiment is a method of treating ulcerative colitis, such as active ulcerative proctitis, in a patient in need thereof by administering to the patient a mesalamine rectal suppository of the present invention. Preferably, the mesalamine suppository is administered once a day and more preferably once a day at bedtime. The suppository is also preferably retained for one to three hours or longer, if possible. The treatment can be brief, for example, once daily for three to twenty-one days, or can be longer, for example, once daily for three to six weeks.

The invention is further illustrated by the following examples which are provided to be exemplary of the invention and do not limit the scope of the invention. While the present invention has been described in terms of its specific embodiments, certain modifications and equivalents will be apparent to those skilled in the art and are intended to be included within the scope of the present invention.

### Example 1

### A. Preparation of Mesalamine Suppository

| Sr. No. | Ingredients | Quantity (% w/w) |
|---|---|---|
| 1 | Mesalamine | 33.34 |
| 2 | Witepsol ™ H-15 | 33.33 |
| 3 | Suppocire ™NA15 | 33.33 |

### B. Procedure

Step-1 Suppository Mixture Preparation

1.1 Melt Hard Fat and add Mesalamine API under stirring at 45° C. in suitable jacketed vessel.

1.2 Continue stirring till uniformly dispersion has resulted.

Step-2 Suppository Filling

2.1 Transfer the molten mass of step 1.2 to the tank of suppository filling m/c and carry out the suppository filling and packing.

### C. Dissolution Test

Torpedo shaped, Light tan colored suppository obtained by above methods were subjected to the dissolution test according to USP Apparatus #2 at 40° C. and a paddle rotation speed of 125 rpm in 0.2 M phosphate buffer at a pH of 7.5.

The results of dissolution test are shown below:

| Time (Min) | Dissolution (% of drug dissolved) |
|---|---|
| 0 | 0 |
| 10 | 45 |
| 20 | 67 |
| 30 | 83 |
| 45 | 92 |
| 60 | 95 |

### Example 2

### A. Preparation of Mesalamine Suppository

| Sr. No. | Ingredients | Quantity (% w/w) |
|---|---|---|
| 1 | Mesalamine | 33.33 |
| 2 | Witepsol ™ H-15 | 26.67 |
| 3 | Suppocire ™NA15 | 40.00 |

### B. Procedure

Step-1 Suppository Mixture Preparation

1.1 Melt Hard Fat and add Mesalamine API under stirring at 45° C. in suitable jacketed vessel.

1.2 Continue stirring till uniformly dispersion has resulted.

Step-2 Suppository Filling

2.1 Transfer the molten mass of step 1.2 to the tank of suppository filling m/c and carry out the suppository filling and packing.

### C. Dissolution Test

Torpedo shaped, Light tan coloured suppository obtained by above methods were subjected to the dissolution test according to USP Apparatus #2 at 40° C. and a paddle rotation speed of 125 rpm in 0.2 M phosphate buffer at a pH of 7.5.

The results of dissolution test are shown below:

| Time (Min) | Dissolution (% of drug dissolved) |
|---|---|
| 0 | 0 |
| 10 | 49 |
| 20 | 62 |
| 30 | 84 |
| 45 | 93 |
| 60 | 97 |

### Example 3

### A. Preparation of Mesalamine Suppository

| Sr. No. | Ingredients | Quantity (% w/w) |
|---|---|---|
| 1 | Mesalamine | 33.33 |
| 2 | Witepsol ™ H-15 | 40.00 |
| 3 | Suppocire ™NA15 | 26.67 |

### B. Procedure

Step-1 Suppository Mixture Preparation

1.1 Melt Hard Fat and add Mesalamine API under stirring at 45° C. in suitable jacketed vessel.

1.2 Continue stirring till uniformly dispersion has resulted.

Step-2 Suppository Filling

2.1 Transfer the molten mass of step 1.2 to the tank of suppository filling m/c and carry out the suppository filling and packing.

### C. Dissolution Test

Torpedo shaped, Light tan coloured suppository obtained by above methods were subjected to the dissolution test according to USP Apparatus #2 at 40° C. and a paddle rotation speed of 125 rpm in 0.2 M phosphate buffer at a pH of 7.5.

The results of dissolution test are shown below:

| Time (Min) | Dissolution (% of drug dissolved) |
|---|---|
| 0 | 0 |
| 10 | 39 |
| 20 | 59 |
| 30 | 80 |
| 45 | 93 |
| 60 | 98 |

### Example 4

### A. Preparation of Mesalamine Suppository

| Sr. No. | Ingredients | Quantity (% w/w) |
|---|---|---|
| 1 | Mesalamine | 33.33 |
| 2 | Witepsol ™ H-15 | 20.00 |
| 3 | Suppocire ™NA15 | 46.67 |

### B. Procedure

Step-1 Suppository Mixture Preparation

1.1 Melt Hard Fat and add Mesalamine API under stirring at 45° C. in suitable jacketed vessel.

1.2 Continue stirring till uniformly dispersion has resulted.

Step-2 Suppository Filling

2.1 Transfer the molten mass of step 1.2 to the tank of suppository filling m/c and carry out the suppository filling and packing.

### C. Dissolution Test

Torpedo shaped, Light tan coloured suppository obtained by above methods were subjected to the dissolution test according to USP Apparatus #2 at 40° C. and a paddle rotation speed of 125 rpm in 0.2 M phosphate buffer at a pH of 7.5.

The results of dissolution test are shown below:

| Time (Min) | Dissolution (% of drug dissolved) |
|---|---|
| 0 | 0 |
| 10 | 47 |
| 20 | 73 |
| 30 | 85 |
| 45 | 96 |
| 60 | 99 |

### Example 5

### A. Preparation of Mesalamine Suppository

| Sr. No. | Ingredients | Quantity (% w/w) |
|---|---|---|
| 1 | Mesalamine | 33.34 |
| 2 | Witepsol ™ H-15 | 33.33 |
| 3 | Suppocire ™NA15 | 33.33 |

### B. Procedure

Step-1 Suppository Mixture Preparation

1.1 Melt Hard Fat and add Mesalamine API under stirring at 45° C. in suitable jacketed vessel.

1.2 Continue stirring till uniformly dispersion has resulted.

Step-2 Suppository Filling

2.1 Transfer the molten mass of step 1.2 to the tank of suppository filling m/c and carry out the suppository filling and packing.

### C. Dissolution Test

Torpedo shaped, Light tan coloured suppository obtained by above methods were subjected to the dissolution test according to USP Apparatus #2 at 40° C. and a paddle rotation speed of 125 rpm in 0.2 M phosphate buffer at a pH of 7.5.

The results of dissolution test are shown below:

| Time (Min) | Dissolution (% of drug dissolved) |
|---|---|
| 0 | 0 |
| 10 | 51 |
| 20 | 72 |
| 30 | 84 |
| 45 | 94 |
| 60 | 99 |

### Example 6

### A. Preparation of Mesalamine Suppository

| Sr. No. | Ingredients | Quantity (% w/w) |
|---|---|---|
| 1 | Mesalamine | 33.33 |
| 2 | Witepsol ™ H-15 | 26.67 |
| 3 | Suppocire ™NA15 | 40.00 |

### B. Procedure

Step-1 Suppository Mixture Preparation

1.1 Melt Hard Fat and add Mesalamine API under stirring at 45° C. in suitable jacketed vessel.

1.2 Continue stirring till uniformly dispersion has resulted.

Step-2 Suppository Filling

2.1 Transfer the molten mass of step 1.2 to the tank of suppository filling m/c and carry out the suppository filling and packing.

### C. Dissolution Test

Torpedo shaped, Light tan coloured suppository obtained by above methods were subjected to the dissolution test according to USP Apparatus #2 at 40° C. and a paddle rotation speed of 125 rpm in 0.2 M phosphate buffer at a pH of 7.5.

The results of dissolution test are shown below:

| Time (Min) | Dissolution (% of drug dissolved) |
|---|---|
| 0 | 0 |
| 10 | 46 |
| 20 | 70 |
| 30 | 83 |
| 45 | 94 |
| 60 | 99 |

### Example 7

### A. Preparation of Mesalamine suppository

| Sr. No. | Ingredients | Quantity (% w/w) |
|---|---|---|
| 1 | Mesalamine | 33.33 |
| 2 | Witepsol ™ H-15 | 40.00 |
| 3 | Suppocire ™NA15 | 26.67 |

### B. Procedure

Step-1 Suppository Mixture Preparation

1.1 Melt Hard Fat and add Mesalamine API under stirring at 45° C. in suitable jacketed vessel.

1.2 Continue stirring till uniformly dispersion has resulted.

Step-2 Suppository Filling

2.1 Transfer the molten mass of step 1.2 to the tank of suppository filling m/c and carry out the suppository filling and packing.

### C. Dissolution Test

Torpedo shaped, Light tan coloured suppository obtained by above methods were subjected to the dissolution test according to USP Apparatus #2 at 40° C. and a paddle rotation speed of 125 rpm in 0.2 M phosphate buffer at a pH of 7.5.

The results of dissolution test are shown below:

| Time (Min) | Dissolution (% of drug dissolved) |
|---|---|
| 0 | 0 |
| 10 | 39 |
| 20 | 67 |
| 30 | 84 |
| 45 | 100 |
| 60 | 104 |

TABLE 1
Particle size distribution of Mesalamine

| Mesalamine PSD by Malvern (Microns) | Examples 1-4 | Examples 5-7 |
|---|---|---|
| D 10 | 3.603 | 3.754 |
| D 50 | 9.302 | 11.108 |
| D 90 | 20.835 | 27.394 |
| API Bulk density (gm/ml) | 0.235 | 0.247 |
| API Tapped density (gm/ml) | 0.427 | 0.446 |

We claim:

1. A pharmaceutical suppository comprising mesalamine or salts thereof and at least two oily or fatty bases, wherein the mesalamine has a tap density ranging from about 250 g/L to about 580 g/L (as measured by USP <616>).

2. The pharmaceutical suppository of claim 1, wherein the mesalamine has a tap density ranging from about 350 g/L to about 550 g/L (as measured by USP <616>).

3. The pharmaceutical suppository of claim 1, wherein the mesalamine has a tap density ranging from about 400 g/L to about 500 g/L (as measured by USP <616>).

4. The pharmaceutical suppository of claim 1, wherein the suppository has a drug load of not more than 35% w/w.

5. The pharmaceutical suppository of claim 4, wherein the drug load ranges from about 33% w/w to 35% w/w.

6. The pharmaceutical suppository of claim 1, wherein the two oily or fatty bases are in ratio from about 1:3 to about 3:1.

7. The pharmaceutical suppository of claim 1, wherein the suppository comprises mesalamine from about 400 to about 1600 mg.

8. The pharmaceutical suppository of claim 1, wherein the oily or fatty base has an ascending melting point of not more than 37° C.

9. The pharmaceutical suppository of claim 1, wherein the suppository releases at least about 75% by weight of the mesalamine contained in the suppository within 2 hours of dissolution as measured with USP Apparatus #2 at 40° C., a paddle rotation speed of 125 rpm, and 3 sinker turns in 0.2 M phosphate buffer at a pH of 7.5.

10. A method of treating active ulcerative proctitis in a patient in need thereof comprising administering the mesalamine suppository of claim 1 to the patient.
